(19) United States
(12) Patent Application Publication
(10) Pub. No.: US 2022/0241210 A1
(43) Pub. Date: Aug. 4, 2022

(54) Polyvalent Dosage Forms and Method For Their Production

(71) Applicant: DiHeSys Digital Health Systems GmbH, Schwäbisch-Gmünd (DE)

(72) Inventors: Markus Dachtler, Ulm (DE); Gerald Huber, Plüderhausen (DE)

(21) Appl. No.: 17/615,559

(22) PCT Filed: May 29, 2020
(86) PCT No.: PCT/EP2020/065098

§ 371 Date: Nov. 30, 2021

(30) Foreign Application Priority Data
May 31, 2019 (EP) 19177757.2

(51) Int. Cl. A61K 9/20; A61K 9/00; A61K 9/24

(52) U.S. Cl. CPC A61K 9/2095; A61K 9/0053; A61K 9/06; A61K 9/209; A61K 9/2018

(57) ABSTRACT

The present invention relates to solid or semi-solid dosage forms made of solidified filament structures having different compositions. At least some of the filament structures contain at least one pharmaceutical active agent, nutraceutical active agent and/or dietary supplemental active agent. Other filament structures of the dosage forms can contain the same active agent e.g., in different concentrations, a different active agent, or no active agent. The invention also relates to a method for 3D printing of the dosage forms.

### FIELD OF THE INVENTION

The present invention relates to solid or semi-solid dosage forms made of solidified filament structures having different compositions. At least some of the filament structures contain at least one pharmaceutical active agent, nutraceutical active agent and/or dietary supplemental active agent. Other filament structures of the dosage forms can contain the same active agent e.g., in different concentrations, a different active agent, or no active agent. The invention also relates to a method for 3D printing of the dosage forms.

### BACKGROUND OF THE INVENTION

The production of dosage forms containing pharmaceutical, nutraceutical or dietary supplemental active agents by fused filament fabrication (FFF; also denoted as filament depositions modelling (FDM)) is known from WO 2016/038356 A1. In a similar process granules, pellets, powders or flakes are used instead of filaments as starting materials which in turn are printed in the form of filaments (fused layer modeling, FLM).

The technical problem of the present invention is the provision of dosage forms and methods for their production by 3D printing, in particular by FFF or FLM, wherein different active agents, combinations of active agents, concentrations of active agents and/or other parameters like e.g., active agent release characteristics can be realized in a single unit.

The above technical problem is solved by the embodiments of the present invention as disclosed in the claims, the present description and the accompanying drawings.

### SUMMARY OF THE INVENTION

The present invention provides a semi-solid or solid dosage form made of solidified filament structures which comprise at least two groups of filament structures, wherein the composition of the filament structures of one group is different from the composition of the filament structures of the other group(s), and at least the filament structures of one group contain at least one active agent selected from the group consisting of pharmaceutical active agents, nutraceutical active agents and dietary supplemental active agents.

Preferred active agents according to the invention are pharmaceutical active agents.

According to the invention “solidified filament structures” are three-dimensional units of a solid or at least semi-solid material or a solid or semi-solid composition, respectively. The filament units typically have a shape resulting from FFF or FLM 3D printing, in particular elongate cylindric, cuboid or prismatic shapes. “Solidified” in the context of the invention means that the semi-solid or solid filament structures have been transformed from an at least flowable form during the production process, particularly as described herein below with respect to the method of the invention, into a solid or semi-solid state. Through deposition of the filament structures during the production in an at least flowable state the deposited filaments are transformed into an at least semi-solid, preferably solid, state, which can occur in a manner known to the skilled person such as by cooling or chemically e.g., by polymerization of the starting substances, particularly polymerizable monomers, or also polymers, contained in the starting composition (hereinafter also denoted as “starting object”). According to the invention, the “solidified filament structures” forming the dosage forms are distinguishable i.e., the individual filament structure can be detected at least by physical (e.g. by optical methods such as microscopic methods) and/or chemical methods although the delimitation of the individual filament structures may be not visible for the human eye. Thus, in the sense of the invention the individual filament structures in the dosage are distinguishable from one another by suitable methods such as e.g., optical methods.

A “group of filament structures” according to the invention are at least two solidified filament structures of essentially same composition or in any case at least two solidified filament structures having one characteristic in common with respect to at least one essential parameter of the invention, in particular contain the same active agent or the same combination of two or more active agents or the same concentration of an active agent, or contain no active agent.

In one embodiment of the invention, the different compositions in the various groups (e.g. two, three, or more groups) can be formed in such a manner that different groups of filament structures contain different active agents or a different composition of active agents (i.e., a combination of 2 or more active agents). Alternatively, the composition of a group of filaments is different from the other group (or the other groups) in that the different groups of filament structures contain the same active agent, but it is present in different concentrations. In other embodiments of the invention, the differences in active agent and concentrations can also be combined e.g., in that several groups of filaments are present wherein e.g., two groups contain the same active agent, but in different concentrations, and a further group contains a different active agent. The different composition of the groups of filaments can also be realized in that one group of filament structures contains an active agent whereas the filament structures of the other group (or another group) contain no active agent. Alternatively, the different groups of filament structures differ in the release characteristic of the active agents or the active agent, respectively, contained therein e.g., in that the release of the active agent(s) is different with respect to release rate, amount released and/or dependence of the release on the surrounding milieu e.g., solubility such as pH-dependent solubility, or low solubility or insolubility such as gastric juice resistance.

As can be derived from the foregoing embodiments, the dosage form of the invention can comprise two or more active agents which can be contained in different filament structures. Dosage forms having 2, 3, 4, 5, 6, 7, or 8 active agents are preferred. Each active agent can be present individually in its own filament structures. However, there can also be combinations of active agents in the filament structures, whereby filament structures having one active agent can be combined in the present invention with filament structures having more than one active agent.

The filament structures of the invention typically comprise the composition used in production processes such as FLM/FFF 3D printing. The filament composition typically contains the components which are described in the following in detail in the context of the method of the invention, optionally including at least on active agent which is selected according to the invention from pharmaceutical, nutraceutical and dietary supplemental active agents. In the following, these active agents are collectively denoted as “active component(s) or also as “active agent(s).

The active components can be selected essentially from any available pharmaceutical, nutraceutical and dietary supplemental active agents being and administrable by semi-solid or solid dosage forms, which agents can in turn be combined freely as long as it is acceptable for the desired application. Preferred dosage forms of the invention contain at least one pharmaceutical active agent in the filament structures of a group, particularly preferred are synergistically acting combinations of 2, 3 or more pharmaceutical active agents which can be present in a single group of filament structures. It is more preferred that the different active agents are contained in filament structures of different groups. Other preferred combinations of 2, 3 or more pharmaceutical active agents are those wherein e.g., a pharmaceutical active agent intended for a certain indication is contained in the dosage form, and a further pharmaceutical active agent is contained in the dosage form, which e.g., reduces or at best suppresses a side effect at least potentially elicited by the first active agent.

Examples of active agents that can be combined according to the invention are agonists/antagonists for reducing the potential of addiction to analgesics (e.g., Tilidine and Naloxone), combined preparations for treatment of e.g., hypertension (preferably combinations of ACE inhibitor and/or calcium channel blocker and/or beta blocker), allergies (e.g., antihistamines and calcium), diarrhea (e.g., mineral substances and Loperamide), prophylaxis of stroke/infarct (anticoagulant and antihypertensive), Parkinson's disease (Levodopa and Benserazide) and analgesics (analgetic and caffeine) etc.

In preferred embodiments of the invention the filament structures having the same active agent or the same composition of active agents or the same concentration of active agent are present in a joint region of the dosage form such that the filament structures at least partially adjoin at least on one side. Thus, it is preferred that the filament structures having the same active agent or the same composition of active agents or the same concentration of active agent form a joint region (e.g., a joint filament layer or at least one connected part of a filament layer, whereby these are preferably arranged horizontally or vertically in relation to the longest dimension of the dosage form). In other embodiments, filament structures having the respective same active agent, same composition of active agents or same concentration of active agent can also be grouped in several regions (thus e.g., 2 or more layers or parts of layers). Such regions can also have different active agent release characteristics such as e.g., different pH solubilities, different gastric juice resistances, other solubility behaviors (e.g., in that the filament structures of certain regions or one certain region contain a burst release substance).

In a preferred variant of this embodiment, regions are arranged horizontally or vertically such that filaments having the same active agent or the same composition of active agents or the same concentration of active agent or the same active agent release characteristic form horizontal or vertical, respectively, layers in the dosage form in relation to the longest dimension of the dosage form. In this embodiment, a region can be formed from only a single layer of filament structures having the same active agent or the same composition of active agents or the same concentration of active agent or the same active agent release characteristic. Alternatively, such region can comprise or consist of several layers of filament structures having the same active agent (or the same composition of active agents or the same concentration of active agent or the same active agent release characteristic). In this embodiment, the regions of filament structures having the same active agent (or the same composition of active agents or the same concentration of active agent or the same active agent release characteristic) can have the same or different dimensions in comparison to regions of filament structures having another active agent or another combination of active agents or another concentration of active agent or another active agent release characteristic. In one embodiment e.g., one or more layers (it can also be partial layers) of filaments having a first active agent (or having a first combination of active agents or having a first concentration of active agent or having a first active agent release characteristic) alternate in a regular or unregular fashion e.g., in horizontal or vertical arrangement (with respect to the longest dimension of the dosage form) with one or more layers of filament structures having a second active agent (or having a second combination of active agents or having a second concentration of active agent or having a second active agent release characteristic).

According to the invention, not necessarily all of the filament structures in the dosage form contain an active component (=active agent). In a preferred embodiment, the filament structures having an active component can e.g., form an active component-containing core of the dosage form, and filament structures not having an active component are provided around the active agent-containing core, preferably such that the active agent-containing core is completely (thus, at least with one layer) surrounded by filament structures not containing an active agent. In other embodiments of the invention, filament structures not having an active agent can also form at least partial, preferably complete, inter layers between filament structures (layers thereof) containing one or more active agents.

In another embodiment, the dosage form comprises at least one partial volume being free of filament structures, said at least partial volume being surrounded by filament structures. The partial volume surrounded by filament structures can be a void not filled with liquid, semi-solid or solid substances (in this case the void typically contains a gas such as air or a different gas such as an inert gas like e.g., nitrogen). In other embodiments, the at least one void at least partially, in preferred embodiments completely, filled with a liquid, semi-solid or solid composition which in certain embodiments can be free of active agent. In other embodiments of the invention, the composition at least partially filling the void contains at least one active agent or a combination of active agents wherein the at least one active agent or the combination of active agents can be the same or different from the active agent(s) or combinations of active agents, respectively, being present in the filament structures. The composition at least partially filling the void is preferably pharmaceutically, nutraceutically or dietary supplementary acceptable.

In a further preferred embodiment, the dosage form contains several, preferably multiple, partial volumes, preferably voids, which are not filled with liquid, semi-solid or solid materials such that the dosage form is in a spongy or porous, respectively, preferably highly porous, form. In one embodiment the voids can also be in connection with the surrounding of the dosage form. Through the number, size and distribution of such voids the surface of the dosage form can be increased significantly, particularly for specifically regulating the amount and/or rate of release of the active agents present in the dosage form (or of the active agent present in the dosage form). In this respect it is also referred to the disclosure content of US 2018/0311169 A1.

In preferred embodiments of the dosage form having several, preferably multiple, voids which are preferably in connection with the surrounding, the surface of the dosage form in comparison to a dosage form being otherwise identical as regards shape and size at least about doubled or more than doubled, such as increased about twofold to about twentyfold, in further preferred embodiments increased about threefold, about fourfold, about fivefold, about sixfold, about sevenfold, about eightfold, about ninefold or about tenfold.

In further embodiments of the dosage form according to the invention filament structures having the same composition, in particular same active agent or same combination of active agents or same concentration of active agent, are arranged in a joint region and separated by a barrier of filament structures having no active agent and/or of filament structures having an active agent being different from said firstly mentioned active agent (or different combination of active agents or different concentration of active agent). Such a barrier can e.g., be a separating layer of a material impermeable for the active agent or several active agents. There can be also separating layers be provided being impermeable for an active agent but being at least partially permeable for another active agent in the dosage form (selective separating layers). Separating layers for use in the dosage form according to the invention can also be impermeable or permeable for other substances in the filament structures.

In preferred embodiments, the barrier (layers) can also be formed by active agent-free filament structures.

The mentioned embodiments for the separation of active agents or other ingredients by a barrier can be used in case e.g., where two active agents, in particular pharmaceutical active agents, are chemically unstable or where an active agent reduces the chemical stability of another active agent, respectively. One combination of active agents in such an embodiment are Aspirin and Clopidogrel. The separation of active agents according to the invention is also preferably embodied in cases where one active agent influences the effectivity of another active agent or where two active agents influence one another's effect. One example of active agents having a mutual influence on their effects is a combination of Aspirin and Ibuprofen. In the mentioned examples, the filament structures containing one of the mentioned active agents are separated in a preferred embodiment of the invention from such filament structures containing the other active agent by a barrier or separating layer, respectively, as described above.

The filament structures of the dosage form according to the invention can be homogeneous, i.e. the dosage form has, except for the optionally contained active agent (or the contained active agents or the contained concentration of active agent) physically and chemically equal characteristics and/or also a homogenous distribution in the dosage form (or at least in parts of the dosage form).

In other embodiments the distribution of density of the filament structures (that is, the number filament structures per unit volume) is not homogenous. This can be attained e.g., by disposing filament structures having different dimensions. In further embodiments of the invention the filament structures have different surface properties (surface textures) such as e.g., smooth, rough, porous, equipped with coatings or similar. By such means, properties such as release kinetics of the active agents, resistance of the dosage form against body fluids such as saliva, gastric juice, certain intestinal fluids can be regulated.

Further embodiments of the invention relate to dosage forms wherein the solidified filament structures having the same composition or at least belonging to the same group in relation to one of the above-mentioned preferred parameters (or at least part of the filament structures having the same composition or the same parameter) are detectably labelled at least on the surface, thus being distinguishable from one another in principle by any method. Such detectable distinguishing features enabling the differentiation of filament structures having the same active agent or the same parameter according to the invention such as those described above, respectively, (or part of the filament structures having the same active agent or parameter, respectively) include e.g., visible dyes, fluorescent dyes, surface textures, shape, gloss, roughness, porosity and similar. Further detectable distinguishing features are those which are distinguishable by absorption or reflection of electromagnetic radiation (such as infrared radiation, ultraviolet light or Raman radiation). In this embodiment these and other distinguishing features can be combined in an essentially free fashion.

The invention also relates to a method for 3D printing of a semi-solid or solid dosage form as described herein by filament fusion fabrication (FFF) or fused layer modelling (FLM) wherein the method comprises the steps of:
providing a printing device designed for FFF or FLM 3D printing;
providing starting objects designed for FLM or FFF 3D printing, wherein the starting objects form at least a first and a second group of starting objects wherein the composition of the starting objects of the first group is different from the composition of the starting objects of the first group, and wherein at least one group of the starting objects contains an active agent selected from the group consisting of pharmaceutical active agents, nutraceutical active agents and dietary supplemental active agents;
printing the starting objects using the printing device until the dosage form is built.

Generally, the method can be carried out using known components of FFF 3D printing or FLM 3D printing, respectively, whereby it can be referred in respect of FFF processes to the respective disclosure of WO 2016/038356 A1.

A “starting object” in the sense of the method according to the invention are materials suitable for FFF 3D printing or FLM 3D printing, respectively, which materials can be processed and printed into an 3D object by principally known printing devices, whereby in the mentioned methods the starting object, or synonymously the starting material, comprises a base composition printable into a filament form. Typically, the starting objects are transformed into a flowable state during the 3D printing process, usually by heating through a heating element present in a printing head, and then deposited through the printing head usually by extrusion in filament form, preferably in layers, onto a building platform. The method according to the invention can also be embodied by printing the starting objects in filament form onto a pre-existing object provided on the building platform.

Thus, the filament structures having been illustrated in the context of the dosage form of the invention are the products of each single printing step of the method according to the invention, which products have been transformed from the flowable state of the starting material, i.e. the starting objects transferred into a flowable state in the printing device, into a semi-solid or solid state.

Starting materials or starting objects, respectively, for a FLM process are typically granules, pellets, powders and/or flakes.

In an embodiment of the method the starting objects of one group contain at least a first active agent or a first composition of active agents and the starting objects of the other or another, respectively, group contain a second active agent or a second composition of active agents, respectively, wherein the second active agent is different from the first active agent and the second composition of active agents is different form the first composition of active agents, respectively.

In a further embodiment of the method the starting objects of the or of another group contain the same active agent wherein the concentration of the active agent in one group is different from the concentration of the active agent in the starting objects of the other group(s). In other words, starting objects or materials are printed in this embodiment forming at least two groups wherein both groups or (in the case of more than two groups) at least two groups, respectively, contain the same active agent but at a different respective concentration. In a further embodiment it is envisaged to provide more than two, preferably 3, 4, 5, 6, 7, or 8, groups of starting objects and to print these into filaments, wherein each group contains an active agent being different from the respective other active agent in the other groups of stating objects. Alternatively, more than one active agent such as 2, 3, or 4 active agents, with 2 active agents being particularly preferred, are present in a group or several groups of starting objects.

As already illustrated above in the context of the dosage form according to the invention, there can also be a group of starting objects be present, i.e. be printed, containing no active agent such that the provided dosage form contains solidified filament structures having no active agent.

In another embodiment of the method the groups of starting objects or materials, respectively, can be provided such that the printed filaments are different with respect to their active agent release characteristics.

It is, of course, possible to combine the aforementioned different characteristics of the starting objects and the printed filaments, respectively.

In a further embodiment of the method each group of starting objects having the same composition (or in which at least one of the aforementioned parameters relevant to the invention is the same) comprises a detectable distinguishing feature being different from that of the other group(s) of starting objects such that the printed filaments having a different composition (or different parameters, respectively) are distinguishable. As already outlined above, preferred distinguishable features are e.g., visible dyes, fluorescent dyes, surface textures, shape, gloss, porosity, roughness, and absorption or reflection characteristic with respect to electromagnetic radiation.

It is preferred that the method is carried out such that one or more layer(s) of the first group of starting objects is/are printed in the form of filaments and subsequently one or more layer(s) of a second group of starting objects are printed in the form of filaments, and optionally, respective further one or more layer(s) of the further groups of starting objects are printed in the form of filaments. The respective groups of starting materials can also be printed as alternate layers.

In a further preferred embodiment of the method according to the invention, the groups of starting objects are printed in a manner such that the generated dosage form has, as described above, at least one partial volume containing no printed filaments. This can be done in a manner known by the person skilled in the art, wherein in one embodiment a void containing air or an inert gas (such as e.g., nitrogen) is formed. In another embodiment, the filaments can also be printed around a (solid or semisolid) composition containing e.g., the same or a different active agent (in comparison to the active agent(s) in the printed filaments). In another preferred embodiment the filaments are printed in manner (e.g., by transverse layering such that discontinuities in the structure of the dosage form arise) such that several, preferably a plurality of, voids are formed which preferably are in connection with the surrounding such that a spongy or porous, respectively, preferably highly porous, dosage form develops.

### BRIEF DESCRIPTION OF THE DRAWINGS

The annexed Figures further illustrate the invention:

FIG. 1 is a photographic representation of an exemplary dosage form of the invention containing five layers having different colors containing a different composition of active agents.

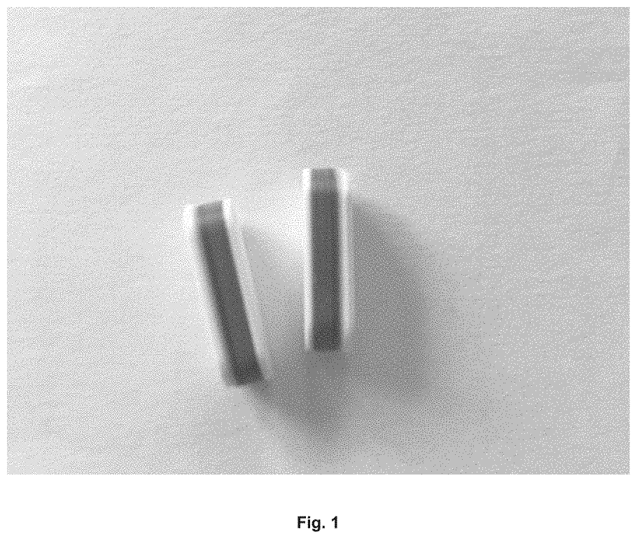

FIG. 2 is a photographic representation of a further exemplary dosage of the invention having a highly porous structure containing voids such that the dosage form has a surface being ca. tenfold higher than a dosage form of identical shape and dimension, but without voids

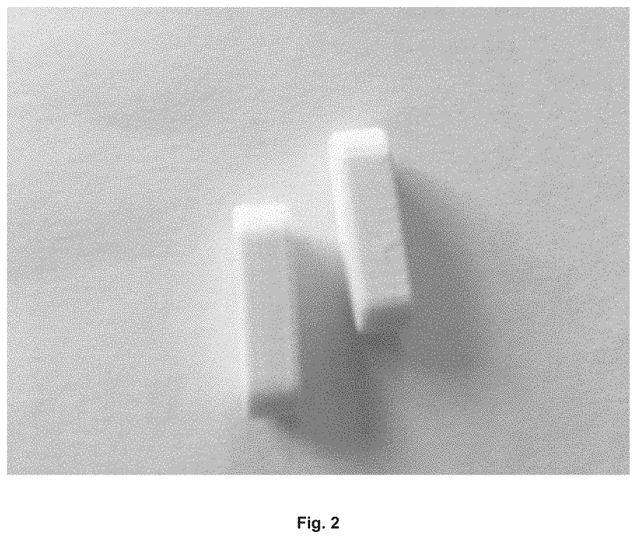

### DETAILED DESCRIPTION OF THE INVENTION

The present invention particularly relates to the following aspects and preferred embodiments:

1. A semi-solid or solid dosage form made of solidified filament structures which comprise at least two groups of filament structures, wherein the composition of the filament structures of one group is different from the composition of the filament structures of the other group(s), and at least the filament structures of one group contain at least one active agent selected from the group consisting of pharmaceutical active agents, nutraceutical active agents and dietary supplemental active agents.
2. The dosage form of point 1 wherein the filament structures of one group contain at least one first active agent or first composition of active agents, and the filament structures of the other or another, respectively, group contain a second active agent or second composition of active agents being different from the first agent or first composition of active agents, respectively.
3. The dosage form of point 1 wherein the filament structures of the other or another group contain the same active agent, wherein the concentration of the active agent in the filament structures of one group is different from the concentration of the active agent in the filament structures of the other group(s).
4. The dosage form according to any one of the preceding points wherein at least one group of filament structures containing no active agent is present.
5. The dosage form according to any one of the preceding points wherein the solidified filament structures having the same composition have at least one distinguishing feature detectable on the surface of the dosage form making these filament structures distinguishable from filament structures having a different composition.
6. The dosage form of point 5 wherein the distinguishing feature is selected from the group consisting of visible dyes, fluorescent dyes, surface textures, shape, gloss, porosity, roughness, and absorption or reflection, respectively, characteristics of electromagnetic irradiation.
7. The dosage form according to any one of the preceding points wherein the filament structures having the same composition are present in a joint region of the dosage form such that the filament structures at least partially adjoin at least on one side.
8. The dosage form of point 7 wherein the regions are arranged horizontally or vertically such that filaments having the same active agent form horizontal or vertical, respectively, layers in the dosage form in relation to the longest dimension of the dosage form.
9. The dosage form according to any one of the preceding points wherein filament structures having the same composition are arranged in a joint region and separated by a barrier of filament structures having no active agent and/or of filament structures having an active agent being different from said active agent and/or of filament structures having the same active agent but a different concentration of said active agent, the barrier being impermeable for said active agent.
10. The dosage form of point 9 wherein the barrier between filament structures having different active agents and/or filament structures having different concentrations of active agent are formed by filament structures having no active agent.
11. The dosage form according to any one of the preceding points wherein the dosage form comprises at least one partial volume being free of filament structures, said at least partial volume being surrounded by filament structures.
12. The dosage form of point 11 wherein the partial volume is a void.
13. The dosage form of point 12 wherein the partial volume is filled with a pharmaceutically acceptable composition containing or not containing active agent.
14. The dosage form of point 13 wherein the composition contains one or more active agent(s) being different from the active agent(s) of the filament structures.
15. The dosage form of point 14 wherein the dosage form comprises multiple voids such that the dosage form is in a spongy form.
16. The dosage form according to any one of the preceding points wherein the dosage form has a an inhomogeneous distribution of density of filament structures.
17. The dosage form according to any one of the preceding points wherein the filament structures in the dosage form have different surface properties.
18. The dosage form according to any one of the preceding points wherein the dosage form contains more than two active agents.
19. The dosage form of point 18 wherein the dosage form contains 3 to 8 active agents.
20. A method for 3D printing of a semi-solid or solid dosage form according to any one of the preceding points by filament fusion fabrication (FFF) or filament layer modeling (FLM) comprising the steps of:
providing a printing device designed for FFF or FLM 3D printing;
providing starting objects designed for FLM or FFF 3D printing, wherein the starting objects form at least a first and a second group of starting objects wherein the composition of the starting objects of the first group is different from the composition of the starting objects of the first group, and wherein at least one group of the starting objects contains an active agent selected from the group consisting of pharmaceutical active agents, nutraceutical active agents and dietary supplemental active agents;
printing the starting objects using the printing device until the dosage form is built.
   21. The method of point 20 wherein the starting objects are selected from the group consisting of filaments, granules, pellets, powder and flakes.
   22. The method of point 20 or 21 wherein the starting objects of one group contain at least a first active agent or a first composition of active agents, and the starting objects of the other or another group contain a second active agent or a second composition of active agents, which second active agent or second composition of active agents, respectively, is different from the first active agent or the first composition of active agents, respectively.
   23. The method of point 20 or 21 wherein the starting objects of the other or another group contain the same active agent, wherein the concentration of the active agent in the starting objects of one group are different from the concentration of the active agent in the other or another group(s).
   24. The method according to any one of points 20 to 23 wherein at least one group of starting objects containing no active agent is present.
   25. The method according to any one of points 20, 21, 22 or 24 wherein more than two groups of starting objects are provided and printed in the form of filaments, wherein each group contains an active agent which is different for the respective active agents of the other groups of starting objects.
   26. The method of point 25 wherein more than 2, preferably 3 to 8, groups of starting objects are provided and printed in the form of filaments.
   27. The method according to any one of points 20 to 26 wherein each group of starting objects of the same composition have a detectable distinguishing feature being different from the other group(s) such that the printed filaments of different composition are distinguishable.
   28. The method of point 27 wherein the distinguishing feature is selected from the group consisting of visible dyes, fluorescent dyes, surface textures, form, gloss, porosity, roughness, and absorption or reflection, respectively, characteristics of electromagnetic irradiation.
   29. The method according to any one of points 20 to 28 wherein one or more layer(s) of the first group of starting objects are printed in the form of filaments and subsequently one or more layer(s) of the second group of starting objects are printed in the form of filaments, and optionally respective one or more layer(s) of the further groups of starting objects are printed as filaments.
   30. The method according to any one of points 20 to 29 wherein the starting objects of the first group and of the second group are in in the form of filaments in different density.
   31. The method according to any one of points 20 to 29 wherein the starting objects of the first and second group of filaments are printed in the form of filaments having different surface properties.

1. A semi-solid or solid dosage form made of solidified filament structures which comprise at least two groups of filament structures, wherein the composition of the filament structures of one group is different from the composition of the filament structures of the other group(s), and at least the filament structures of one group contain at least one active agent selected from the group consisting of pharmaceutical active agents, nutraceutical active agents and dietary supplemental active agents.

2. The dosage form of claim 1 wherein the filament structures of one group contain at least one first active agent or first composition of active agents, and the filament structures of the other or another, respectively, group contain a second active agent or second composition of active agents being different from the first agent or first composition of active agents, respectively.

3. The dosage form of claim 1 wherein the filament structures of the other or another group contain the same active agent, wherein the concentration of the active agent in the filament structures of one group is different from the concentration of the active agent in the filament structures of the other group(s).

4. The dosage form according to claim 1, wherein at least one group of filament structures containing no active agent is present.

5. The dosage form according claim 1, wherein the solidified filament structures having the same composition have at least one distinguishing feature detectable on the surface of the dosage form making these filament structures distinguishable from filament structures having a different composition.

6. The dosage form of claim 5 wherein the distinguishing feature is selected from the group consisting of visible dyes, fluorescent dyes, surface textures, shape, gloss, porosity, roughness, and absorption or reflection, respectively, characteristics of electromagnetic irradiation.

7. The dosage form according to claim 1, wherein the filament structures having the same composition are present in a joint region of the dosage form such that the filament structures at least partially adjoin at least on one side.

8. The dosage form of claim 7 wherein the regions are arranged horizontally or vertically such that filaments having the same active agent form horizontal or vertical, respectively, layers in the dosage form in relation to the longest dimension of the dosage form.

9. The dosage form according to claim 1, wherein filament structures having the same composition are arranged in a joint region and separated by a barrier of filament structures having no active agent and/or of filament structures having an active agent being different from said active agent and/or of filament structures having the same active agent but a different concentration of said active agent, the barrier being impermeable for said active agent.

10. The dosage form of claim 9 wherein the barrier between filament structures having different active agents and/or filament structures having different concentrations of active agent are formed by filament structures having no active agent.

11. The dosage form according to claim 1, wherein the dosage form comprises at least one partial volume being free of filament structures, said at least partial volume being surrounded by filament structures.

12. The dosage form of claim 11 wherein the partial volume is a void.

13. The dosage form of claim 12 wherein the partial volume is filled with a pharmaceutically acceptable composition containing or not containing active agent.

14. The dosage form of claim 13 wherein the composition contains one or more active agent(s) being different from the active agent(s) of the filament structures.

15. The dosage form of claim 14 wherein the dosage form comprises multiple voids such that the dosage form is in a spongy form.

16. The dosage form according to claim 1, wherein the dosage form has an inhomogeneous distribution of density of filament structures.

17. The dosage form according to claim 1, wherein the filament structures of one group and the filament structures of the other group in the dosage form have different surface properties.

18. The dosage form according to claim 1, wherein the dosage form contains more than two active agents.

19. The dosage form of claim 18 wherein the dosage form contains 3 to 8 active agents.

20. A method for 3D printing of a semi-solid or solid dosage form by filament fusion fabrication (FFF) or filament layer modeling (FLM) comprising the steps of:
providing a printing device designed for FFF or FLM 3D printing;
providing starting objects designed for FLM or FFF 3D printing, wherein the starting objects form at least a first and a second group of starting objects wherein the composition of the starting objects of the first group is different from the composition of the starting objects of the first group, and wherein at least one group of the starting objects contains an active agent selected from the group consisting of pharmaceutical active agents, nutraceutical active agents and dietary supplemental active agents;
printing the starting objects using the printing device until the dosage form is built.

21. The method of claim 20 wherein the starting objects are selected from the group consisting of filaments, granules, pellets, powder and flakes.

22. The method of claim 20, wherein the starting objects of one group contain at least a first active agent or a first composition of active agents, and the starting objects of the other or another group contain a second active agent or a second composition of active agents, which second active agent or second composition of active agents, respectively, is different from the first active agent or the first composition of active agents, respectively.

23. The method of claim 20, wherein the starting objects of the other or another group contain the same active agent, wherein the concentration of the active agent in the starting objects of one group are different from the concentration of the active agent in the other or another group(s).

24. The method according to claim 20, wherein at least one group of starting objects containing no active agent is present.

25. The method according to claim 20, wherein more than two groups of starting objects are provided and printed in the form of filaments, wherein each group contains an active agent which is different for the respective active agents of the other groups of starting objects.

26. The method of claim 25 wherein more than 2, preferably 3 to 8, groups of starting objects are provided and printed in the form of filaments.

27. The method according to claim 20, wherein each group of starting objects of the same composition have a detectable distinguishing feature being different from the other group(s) such that the printed filaments of different composition are distinguishable.

28. The method of claim 27 wherein the distinguishing feature is selected from the group consisting of visible dyes, fluorescent dyes, surface textures, form, gloss, porosity, roughness, and absorption or reflection, respectively, characteristics of electromagnetic irradiation.

29. The method according to claim 20, wherein one or more layer(s) of the first group of starting objects are printed in the form of filaments and subsequently one or more layer(s) of the second group of starting objects are printed in the form of filaments, and optionally respective one or more layer(s) of the further groups of starting objects are printed as filaments.

30. The method according to claim 20, wherein the starting objects of the first group and of the second group are in in the form of filaments in different density.

31. The method according to claim 20, wherein the starting objects of the first and second group of filaments are printed in the form of filaments having different surface properties.
